(19) United States
(12) Patent Application Publication
(10) Pub. No.: US 2004/0029800 A1
(43) Pub. Date: Feb. 12, 2004

(54) Remedies for obesity and obesity-associated diseases and method of evaluating these remedies

(76) Inventor: Tomoaki Hoshino, Fukuoka (JP)

Correspondence Address: MERCHANT & GOULD PC, P.O. BOX 2903, MINNEAPOLIS, MN, 55402-0903, US

(21) Appl. No.: 10/381,498

(22) PCT Filed: Oct. 15, 2001
(86) PCT No.: PCT/JP01/09022

(30) Foreign Application Priority Data
Oct. 16, 2000 (JP) 2000-315323

(51) Int. Cl. A61K 38/17

(52) U.S. Cl. 514/12

(57) ABSTRACT

The present invention relates to remedies for obesity and obesity-related diseases which comprise as an active ingredient STAT6 having an effect of accelerating the signal transduction pathway from IL-13 to STAT6, or a related substance thereof; and a method of evaluating the activity of such a remedy against obesity and obesity-related diseases by administering the test drug to a STAT6 knockout non-human animal.

### TECHNICAL FIELD

The invention relates to remedies for obesity and obesity-related diseases which comprise as active ingredient STAT6 or a related substance thereof; and a method of evaluating said remedies.

### BACKGROUND ART

Obesity has been highlighted as a great social problem because it may cause various diseases. Diseases caused by obesity include ones which have considerable impact on people's lives such as diabetes, hypertension, hyperlipidemia, nephropathy and atherosclerosis, and therefore, a remedy for obesity has been urged to prevent and/or treat these diseases.

Diabetes are divided roughly into Type I (insulin dependent diabetes) and Type II (non-insulin dependent diabetes) and more than 90% of diabetes patients are classified into Type II. In contrast to Type I diabetes (juvenile-onset), Type II diabetes usually develops with obesity in middle and aged persons. Thus, obesity is the most important factor for developing Type II diabetes which constitutes a majority of diabetes. The rate of the obesity has been increasing under the current social environment, which has led to increased number of patients with Type II diabetes. In addition, a significant number of the patients evolve into insulin resistant diabetes.

As a result of advance in investigation of molecular mechanisms for obesity, a signal transduction pathway, which a obesity-regulating factor secreted by a adipose cell, leptin, acts on, has drawn attention as an approach for developing obesity drug, and an attempt was made to develop such drug with leptin.

However, leptin per se proved difficult to apply to obesity drug because leptin resistance occurs by administration of leptin. Instead, ciliary neurotrophilic factor (CNTF) which has a function to activate the leptin-acting signal transduction pathway and which does not induce leptin resistance has been drawn attention. Neuropeptide (NPY), which is a downstream factor of leptin, and melanocortin (MC) 4 receptor have also attracted attention.

It was reported that leptin was activated with STAT3, STAT5 and STAT6 [Proc. Natl. Acad. Sci. USA, 93, 6231-6235 (1996)]. However, the ensuing findings that STAT6 knockout mouse develops no rapid obesity unlike leptin knockout mouse (ob/ob mouse) or leptin receptor knockout mouse (db/db mouse) revealed-that at least STAT6 plays no important role in leptin signal transduction system [Immunity, 7, 1-11 (1997)].

JAK-STAT pathway-forming STAT family which was found in cytokine signal transduction pathway reportedly now falls into 7 classes, STAT1, STAT2, STAT3, STAT4, STAT5a, STAT5b and STAT6, and its physiological role has been clarified by using the knockout mouse.

Thus, STAT6 knockout mouse has demonstrated that STAT6 has an intimate involvement in interleukin-4 (IL-4) and interleukin-13 (IL-13) signal transduction pathways [Immunity. 7, 1-11 (1997)]. However, it has not been known that STAT6 knockout mouse develops obesity thereby developing Type II diabetes. Instead, as described above, it has been reported that STAT6 knockout mouse develops no obesity.

### DISCLOSURE OF THE INVENTION

An aspect of the present invention is to provide a new drug used for treating obesity and obesity-related diseases and an evaluation method thereof.

The present inventor conducted a long-term observation of STAT6 knockout mouse and found, contrary to the previous knowledge, that STAT6 knockout mouse develops obesity with aging thereby developing Type II diabetes. The occurrence of increased blood insulin levels and hypertrophy of the pancreas islet tissue also revealed that STAT6 knockout mouse develops insulin resistant Type II diabetes.

In the above experiment, there also found no difference in food consumption between STAT6 knockout mouse and normal mouse. This shows that said obesity was not a result of hyperphagia. Since it is considered that feeding is controlled by leptin, leptin signal transduction system is confirmed to normally function in STAT6 knockout mouse.

According to the above findings, the present inventor prepared a knockout mouse against a cytokine which acts on STAT6 signal transduction system, with which various studies were made, and found that IL-13 and STAT6 signal molecules are deeply involved in obesity, thereby accomplished the present invention.

The present inventor considered, on the basis of the above findings, that evaluation of activity of a test agent on obesity and obesity-related diseases by administering the test agent to a STAT6 knockout non-human animal would enable the evaluation of an agent effective to those diseases, according which a method of evaluating a remedy for said diseases has been accomplished.

Thus, the present invention relates to remedies for obesity and obesity-related diseases which comprise as an active ingredient STAT6 or a related substance thereof and a method of evaluating said remedies.

Hereinafter, the present invention will be descrived in more detail.

### BEST MODE FOR CARRYING OUT THE INVENTION

STAT6 and a related substance thereof which are used for the present invention include, but are not limited to, a substance enabling to activate function of STAT6, i.e., a substance enabling to accelerate signal transduction pathway from IL-13 to STAT6.

STAT6 and a related substance thereof which are used for the present invention include for example STAT6 (protein), IL-13 (protein) and IL-13 receptor (protein).

Preferred STAT6, IL-13 and IL-13 receptor which are used for the present invention are human STAT6, IL-13 and IL-13 receptors, respectively.

STAT6 which is used for the present invention is known and can be prepared according to a method as described in, for example, Molecular and Cellular Biology, vol. 15, No. 6, 3336-3343 (1995).

IL-13 which is used for the present invention is also known and can be prepared according to methods as described in, for example, Kohyo (Jpn. Unexamined Patent Publication) No. H-11-505247 and a reference [Proc. Natl. Acad. Sci. USA, Vol. 90, 3735-3739 (1993)].

IL-13 receptor which is used for the present invention is also known and can be prepared according to a method as described in, for example, Kohyo (Jpn. Unexamined Patent Publication) No. H11-511028.

IL-13 receptor which is used for the present invention may be either IL-13 receptor α or IL-13 receptor β. These receptors are also described in the above Kohyo (Jpn. Unexamined Patent Publication) No. H11-511028. The publication also discloses purified polypeptide and a physiologically active fragment thereof, both of which can be used as a STAT6-related substance.

Remedies for obesity and obesity-related diseases of the present invention can be prepared by using the above STAT6 or a related substance thereof as an effective ingredient and, if appropriate, adding a pharmaceutical additive, and then, preparing in various formulation forms such as injection, suppository, sublingual tablet, inhaler, or implantable preparation by conventional means.

For example, injection can be prepared according to conventional means, which can be prepared by suitably adding an isotonizing agent, such as mannitol, sodium chloride, glucose, sorbitol, glycerol, xylitol, fructose, maltose, and mannose; a stabilizer such as sodium sulfite and albumin; and a preservative such as benzyl alcohol, methyl parahydroxybenzoate, to STAT6 or a related substance thereof.

The injection can also be formulated as a lyophilized preparation which is reconstituted when using. The lyophilized preparation can be prepared in the conventional means, to which the foregoing isotonizing agent, stabilizer, preservative and the like can be properly added.

A remedy for obesity and obesity-related diseases of the present invention can be orally or parenterally administered via said preparations.

Dose of remedy for obesity and obesity-related diseases of the present invention, which may vary depending on the form and administration route of STAT6 or a related substance thereof used for the present invention, and sexuality, pathology, age, body weight and the like of a patient, is usually 0.1 mg to 500 mg per day, which is properly administered as single dose or in 2-3 divided doses.

A method of evaluating a test substance against obesity and obesity-related diseases included in the present invention can be performed by administering the test substance to a STAT6 knockout non-human animal to evaluate activity of the test substance on obesity and obesity-related diseases.

A method for evaluation of the present invention is preferably used for evaluating an agent effective to Type II diabetes-complicated obesity, particularly obesity complicated with insulin resistant diabetes. STAT6 knockout non-human animal used for an evaluation method of the present invention is preferably rodent such as a mouse and rat because of ease of preparation and handling, with particularly mouse being preferred. In addition, a male non-human animal is preferably used (see the following examination examples).

Specifically, an evaluation method of the present invention can be conducted as follows.

That is, it can be conducted by properly administering a test substance to STAT6 knockout non-human animal developing said disease, which was prepared as described in the following Examination 1 (mouse is used in Examination 1), and then, weighing the effect of said test substance on increase in body weight and abnormal sugar metabolism on the basis of sugar tolerance test, after administration of the test substance (vs. agent non-administration group).

The effect of the present invention will be apparent from the result of Examination 1.

### Examination 1

Demonstration of Obesity and Development of Type II Diabetes in STAT6 Knockout Mouse

(1) Examination Method:

C57BL/6 (B6) STAT6 knockout (STAT6 KO) mice (4 male and female animals per each group) were kept up to 47-week old, weighed with time, followed by Intraperitoneal Glucose Tolerance Test (IPGTT) according to a method as described in a reference [DIABETES, VOL., 31, No. 9, 749-753 (1982)].

Thus, glucose tolerance test was conducted by intraperitoneally administering glucose [1 mg/g of body weight] after 6-9 hours fasting, bleeding 0, 30 and 60 minutes after administration, and determining glucose levels in the obtained blood sample by glucose-oxydase method.

In addition, as a control, normal B6 mice (5 male and female animals per each group) were tested in the similar manner as the above experiment.

Used were STAT6 knockout mice which were prepared by using B6-derived ES cells according to a method as described in a reference (Nature 380, 627-630, 1996).

(2) Results of the Examination:

The results were shown in Table 1.

TABLE I

| | | |
|---|---|---|
| | Mean | |
| Group | Body | Glucose Tolerance Test |
| (40-week | Weight | Mean Glucose Levels (mg/dl) |

| old) | (g) | 0 min | 30 min | 60 min |
|---|---|---|---|---|
| STAT6 KO | 40.5 | 257.3 | 532.0 | 597.8 |
| Male | | | | |
| Normal | 31.7 | 179.0 | 235.4 | 196.0 |
| Male | | | | |
| STAT6 KO | 28.8 | 149.8 | 205.0 | 203.0 |
| Female | | | | |
| Normal | 28.1 | 158.4 | 180.2 | 178.8 |
| Female | | | | |

As shown in Table 1, it was confirmed that STAT6 knockout mice grew corpulent compared with control group (normal group), and consequently developed Type II diabetes accompanied by symptoms such as age-related obesity, significant increase in fasting blood sugar, abnormal sugar tolerance, and sugar urine.

In particular, these symptoms were significantly observed in male STAT6 knockout mice.

Thus, male STAT6 knockout mice group developed obesity at 8-week old, abnormal sugar tolerance at 12-week old, significant increase in fasting blood sugar at 30-week old. On the other hand, female STAT6 knockout mice group showed normal sugar metabolism in 30-week old, and mildly abnormal sugar tolerance at 40-week old. The fact suggested that sexual specificity, possibly sex hormone, would be involved in the development of obesity and abnormal sugar tolerance, and onset of diabetes.

In the above experiment, food consumption in 15-week old male normal mice and STAT6 knockout mice was determined for 3 days. The food consumption was 2.60±0.33 g/mouse/day for normal mice and 2.72±0.27 g/mouse/day for STAT6 knockout mice. Thus, there found no difference in the consumption between STAT6 knockout mice and normal mice. This shows that the development of the obesity is not a result of overeating. In addition, since leptin is considered to control feeding, leptin signal transduction system for feeding was confirmed to normally act on STAT6 knockout mice.

In addition, blood glucose level in STAT6 knockout mice increased with aging compared with that in normal mice. In particular, this symptom was significantly observed in male STAT6 knockout mice (Table 2). Enlarged pancreas islet tissue was also confirmed in STAT6 knockout mice.

TABLE 2

Mean Serum Insulin Level (pg/ml)

| Group | 9 weeks | 15 weeks | 22 weeks | 45 weeks |
|---|---|---|---|---|
| STAT6 KO | 504.0 | 1299.1 | 1446.1 | 1754.4 |
| Male | | | | |
| Normal | 402.9 | 382.0 | 1164.5 | 1234.5 |
| Male | | | | |
| STAT6 KO | 431.2 | 672.2 | 484.9 | 317.7 |
| Female | | | | |
| Normal | 245.3 | 303.5 | 375.1 | 453.5 |
| Female | | | | |

The results mentioned above demonstrated that Type II diabetes developed in the aged STAT6 knockout mice is insulin resistant, i.e., those mice having developed insulin resistant diabetes. Accordingly, STAT6 signal transduction pathway was found to be deeply involved in the development of insulin resistant diabetes.

On the basis of the above experimental results, the inventor prepared IL-4 knockout mice, IL-13 knockout mice, and IL-4/IL-13 double knockout mice, in which the factors were estimated to relate to STAT6 signal transduction pathway, with which a similar examination as described above was conducted, and found that no obesity was observed in IL-4 knockout mice but obesity or tendency to develop obesity was observed in IL-13 knockout mice and IL-4/IL-13 double knockout mice. In this case, obesity and abnormal sugar metabolism were observed in male animals as is the case with male STAT6 knockout mice. However, there found neither significant obesity nor abnormal sugar metabolism in female mice.

IL-4 knockout mice, IL-13 knockout mice, and IL-4/IL13 double knockout mice used for the above examination were prepared according to a method as described in references [J Exp Med, 184, 1651-1661, 1996, and J Exp Med, 189, 1565-1572, 1999].

The above findings demonstrated that signal transduction pathway from IL-13 to STAT6 is deeply involved in obesity. However, there found sexual specificity in this signal transduction pathway.

Accordingly, it is considered that acceleration of signal transduction pathway from IL-13 to STAT6 leads to alleviation of obesity, thereby bringing about preventive and/or therapeutic effects on diabetes, nephropathy, retinopathy, neuropathy, hypertension, hyperlipidemia, and atherosclerosis, of which development may be triggered by obesity. In particular, accelerating STAT6 signal transduction pathway is considered to be useful for treating Type II diabetes, particularly obesity complicated insulin resistant diabetes.

The present invention will be concretely described by the following examples.

### EXAMPLE 1

Injection:

Injection containing human IL-13 (5 mg/ample) is prepared by mechanically sterilizing PBS in which human IL-13 is dissolved (1 mg/ml), followed by dispensing 5 ml each per ampule.

### EXAMPLE 2

Injection:

Injection containing human IL-13 receptor (5 mg/ample) is prepared by mechanically sterilizing PBS in which human IL-13 receptor is dissolved (1 mg/ml), followed by dispensing 5 ml each per ampule.

### EXAMPLE 3

Injection:

Injection containing human STAT6 (5 mg/ample) is prepared by mechanically sterilizing PBS in which human STAT6 is dissolved (1 mg/ml), followed by dispensing 5 ml each per ampule.

### EXAMPLE 4

Injection:

Lyophilized injection containing human IL-13 (1 mg/vial) is prepared by dissolving human IL-13 into saline with 5% (W/V) glucose (1 mg/ml), and after mechanical sterilization dispensing 1 ml each per vial.

### EXAMPLE 5

Injection:

Lyophilized injection containing human IL-13 receptor (1 mg/vial) is prepared by dissolving human IL-13 receptor into saline with 5% (W/V) glucose (1 mg/ml), and mechanically sterilizing, followed by dispensing 1 ml each per vial.

### EXAMPLE 6

Injection:

Lyophilized injection containing human STAT6 (1 mg/vial) is prepared by dissolving human STAT6 into saline with 5% (W/V) glucose (1 mg/ml), and mechanically sterilizing, followed by dispensing 1 ml each per vial.

### Industrial Applicability

Remedies for obesity and obesity-related diseases of the present invention accelerate signal transduction pathway from IL-13 to STAT6, which was demonstrated to be involved in obesity by the present inventor, i.e., a signal transduction pathway, wherein IL-13 binds to IL-13 receptor to activate JAK1 assembled to intracellular region of said receptor, the activated JAK1 tyrosine-phosphorylates a transcription factor, STAT6, which then moves into the nucleus to develop induction of transcription of a specific gene, to alleviate obesity, thereby providing protective effect and/or therapeutic effect on such as diabetes, nephropathy, retinopathy, neurosis, hypertension, hyperlipidemia, and atherosclerosis, of which development may be triggered by obesity. Particularly, remedies for obesity and obesity-related diseases of the present invention are effective to obesity which is complicated Type II diabetes, especially, insulin resistant diabetes.

In addition, a method of evaluating a test substance for obesity and obesity-related diseases allows to appropriately evaluate a substance effective to obesity complicated with insulin resistant diabetes, thereby a substance effective to said diseases can be selected from the test substances.

We claim:

1. A remedy for obesity and obesity-related diseases characterized by comprising STAT6 or a related substance thereof as an active ingredient.

2. A remedy as claimed in claim 1 wherein the obesity and obesity-related diseases are obesity complicated with Type II diabetes.

3. A remedy as claimed in claim 2 wherein the Type II diabetes is insulin resistant diabetes.

4. A remedy as claimed in any one of claims 1 to 3 wherein human STAT6 is used as STAT6.

5. A remedy as claimed in any one of claims 1 to 3 wherein the STAT6-related substance is a substance activating function of STAT6.

6. A remedy as claimed in claim 5 wherein interleukin-13 is used as the substance activating function of STAT6.

7. A remedy as claimed in claim 6 wherein human interleukin-13 is used as the interleukin-13.

8. A remedy as claimed in claim 5 wherein interleukin-13 receptor is used as the substance activating function of STAT6.

9. A remedy as claimed in claim 8 wherein human interleukin-13 receptor is used as the interleukin-13 receptor.

10. Use of STAT6 or a related substance thereof for preparing a remedy for obesity and obesity-related diseases.

11. Use of STAT6 or a related substance thereof as claimed in claim 10 wherein the remedy for obesity and obesity-related diseases is the one for obesity complicated with Type II diabetes.

12. Use of STAT6 or a related substance thereof as claimed in claim 11 wherein the Type II diabetes is insulin resistant diabetes.

13. Use of STAT6 or a related substance thereof as claimed in any one of claims 10 to 12 wherein human STAT6 is used as STAT6.

14. Use of STAT6 or a related substance thereof as claimed in any one of claims 10 to 12 wherein STAT6-related substance is the substance activating function of STAT6.

15. Use of STAT6 or a related substance thereof as claimed in claim 14 wherein interleukin-13 is used as the substance activating function of STAT6.

16. Use of STAT6 or a related substance thereof as claimed in claim 15 wherein human interleukin-13 is used as the interleukin-13.

17. Use of STAT6 or a related substance thereof as claimed in claim 14 wherein interleukin-13 receptor is used as the substance activating function of STAT6.

18. Use of STAT6 or a related substance thereof as claimed in claim 17 wherein human interleukin-13 receptor is used as the interleukin-13 receptor.

19. A method of evaluating activity of a test substance on obesity and obesity-related diseases by administering said substance to a STAT6 knockout non-human animal.

20. A method for evaluation as claimed in claim 19 wherein the obesity and obesity-related diseases are obesity complicated with Type II diabetes.

21. A method for evaluation as claimed in claim 20 wherein the Type II diabetes is insulin resistant diabetes.

22. A method for evaluation as claimed in claim 19 wherein the non-human animal is a rodent animal.

23. A method for evaluation as claimed in claim 22 wherein the rodent animal is mouse.

24. A method for evaluation as claimed in any one of claims 19 to 23 wherein a male animal is used as the non-human animal.
